(19) United States
(12) Patent Application Publication
(10) Pub. No.: US 2025/0290058 A1
(43) Pub. Date: Sep. 18, 2025

(54) METHOD FOR PURIFYING TETANUS TOXIN OR VARIANTS THEREOF

(71) Applicants: SHANGHAI REINOVAX BIOLOGICS CO. LTD, Shanghai (CN); SHANGHAI MICRODOM BIOTECH CO. LTD, Shanghai (CN)

(72) Inventors: Juanjuan Wang, Shanghai (CN); Jeff Xianchao Zhu, Shanghai (CN); Xishuang Xiong, Shanghai (CN); Yizhi Mao, Shanghai (CN); Enhua Shen, Shanghai (CN); Qingfeng Xia, Shanghai (CN); Xiaomin Huang, Shanghai (CN); Chang Liu, Shanghai (CN)

(21) Appl. No.: 18/605,800

(22) Filed: Mar. 14, 2024

(51) Int. Cl. C12N 9/52; B01D 15/20; B01D 15/36; B01D 15/38

(52) U.S. Cl. CPC C12N 9/52; B01D 15/203; B01D 15/363; B01D 15/3804; C12Y 304/24068

(57) ABSTRACT

The present disclosure discloses a method for purifying tetanus toxin or variants thereof. The method for purifying tetanus toxin or variants thereof uses metal chelate affinity chromatography, and the tetanus toxin does not contain an affinity tag. The present disclosure uses biocompatible agarose-based nickel affinity chromatography and anion exchange chromatography as separation subjects, which can quickly and effectively remove impure proteins and enrich target proteins; it improves the separation and purification efficiency of proteins and greatly reduces the loss of proteins.

### STATEMENT REGARDING PRIOR DISCLOSURES BY AN INVENTOR OR JOINT INVENTOR

The contents of Chinese Patent Application No. 202210605902.2, filed on May 30, 2022 and published on Sep. 12, 2023, is a grace period disclosure and shall not be prior art to claimed invention.

### REFERENCE TO SEQUENCE LISTING

This application includes a Sequence Listing filed electronically as an XML file named “Sequence Listing_BSHING-23137-USPT.xml”, created on Mar. 13, 2024, with a size of 16 KB. The Sequence Listing is incorporated herein by reference.

### TECHNICAL FIELD

The present disclosure belongs to the field of protein purification, in particular relates to a method for purifying tetanus toxin or variants thereof.

### BACKGROUND

Tetanus toxin has a full length of 1315 amino acids with a relative molecular weight of approximately 150 kDa, and consists of three structural domains: A, B, and C. Fragment A, i.e. a light chain, serves as the active part of the toxin, with a molecular weight of approximately 50 kDa; a heavy chain consists of two fragments, B and C, with a molecular weight of approximately 100 kDa, and the binding site between the toxin and gangliosides is located on the heavy chain. The light chain and heavy chain are connected by disulfide bonds and non-covalent interactions. Tetanus toxin fragment C (here named as TTr protein), located at the C-terminus of the heavy chain, with a relative molecular weight of 50 kDa, serves as the receptor binding region of the toxin, which lacks toxicity, exhibits good immunogenicity, has lower allergenicity than that of tetanus toxoid, and is thus a potential tetanus vaccine antigen. In addition, the TTr protein can also be used as a carrier protein for the development of conjugate vaccines, and it is also a potential carrier for neurotargeted drugs.

Tetanus toxin fragment C contains four cysteine residues in the molecule, which is prone to form intermolecular and intramolecular disulfide bonds, leading to the formation of monomers, dimers and polymers. Moreover, these different conformations can undergo interconversion with different protein concentrations, ambient temperatures, and buffer conditions, which poses certain challenges in the vaccine production process. To address this issue, key cysteine residues in the TTr protein can be mutated to construct conformationally stable TTr protein variants, laying the foundation for the preparation of conformationally stable tetanus subunit vaccines.

The literature “Study on Purification of Tetanus Toxin by Chromatography Procedures” introduced the purification of tetanus toxin using hydrophobic chromatography and ion exchange chromatography; the literature “Purification of Tetanus Toxin and Its Fragment” reported the preparation of the toxin using ammonium sulfate precipitation and gel filtration. There are many reports on separation and methods for purifying tetanus toxin fragment C, among which the patent “High-level Expression of Tetanus Toxin Receptor Binding Domain Hc in Escherichia coli and Application” reported that after a three-step purification using QFF column, phenyl hydrophobic column, and SP column, the purity of the obtained target protein reached 95% with a yield of approximately 300 mg/L; the literature (“Purification and Immunogenicity of Recombinant Tetanus Toxin Fragment C”, 2011, 24 (12), 1430-1433) introduced a three-step purification using ammonium sulfate precipitation, hydrophobic chromatography (Phenyl Sepharose Fast Flow), and anion exchange chromatography (DEAE Sepharose Fast Flow), the purity of the target protein reached 96.7%; in the literature (“Prokaryotic Expression, Purification and Activity of Recombinant Tetanus Toxin Fragment C”, 2010, 23 (3), 270-273), the target protein was obtained by a two-step purification using ion exchange and gel filtration chromatography; in the literature (“Production and Immunogenicity Analysis of Conformation-Stable Fragment Hc Mutant (HcM) of Tetanus Toxin”, 2011, 27 (2): 226-232), the target protein with a purity of 95% was obtained by a three-step purification using ion exchange (QXL) column, hydrophobic chromatography (phenyl-Hs) column, and ion exchange (Source 30Q) column.

Metal chelate affinity chromatography, also known as immobilized metal ion affinity chromatography (IMAC), is currently a widely used technique for the separation and purification of recombinant proteins with affinity tags (e.g., 6×His tag). Its principle is to use multiple amino acids (e.g., histidine) on the protein surface to undergo special coordination binding with transition metal ions (e.g., Ni²⁺, Cu²⁺, Zn²⁺, Co²⁺, etc.), thereby achieving specific purification and separation. In the process of preparing recombinant protein, in order to facilitate purification, it is common to add tags that specifically chelate metal ions, such as 6×His, to the N-terminus or C-terminus in the construction of the expressed genes for the purpose of rapid purification of recombinant proteins with His tags.

When TTr proteins and variants thereof are used in drug research, the recombinant proteins designed for expression usually do not contain affinity tags. Existing literature reports that the purification of TTr proteins without affinity tags is generally obtained by multi-step chromatography, such as hydrophobic chromatography, anion exchange chromatography, and gel filtration chromatography. Currently, the methods for purifying tetanus toxin and variants thereof are inefficient, with a low product yield, which do not meet the requirements for industrial-scale production.

### SUMMARY

Currently, the methods for purifying tetanus toxin and variants thereof are characterized by poor specificity, multiple influencing factors, difficult in condition optimization, low purification efficiency, and low product yield, thus greatly increasing production costs and difficulties in process control.

In order to solve the technical problems described above, the present disclosure provides a method for purifying tetanus toxin or variants thereof. In their research, the inventors discovered for the first time that tetanus toxin, TTr protein, or variants thereof without affinity tags can interact with the medium of the metal chelate affinity chromatography, and can selectively adsorb the proteins, so that tetanus toxin, TTr protein or variants thereof can be rapidly isolated by metal chelate affinity chromatography with high purity and high recovery rate.

The present disclosure uses biocompatible agarose-based nickel affinity chromatography and anion exchange chromatography as separation subjects, which can quickly and effectively remove impure proteins and enrich target proteins; it improves the separation and purification efficiency of the target protein and greatly reduces the loss of the target protein, which is more conducive to large-scale production.

A first aspect of the present disclosure provides a method for purifying tetanus toxin or variants thereof, comprising using metal chelate affinity chromatography to purify tetanus toxin or variants thereof, wherein the tetanus toxin does not contain an affinity tag. For example, it does not contain a tag for purification, which is conventional in the art such as histidine tag (6×His) or GST tag.

Preferably, the tetanus toxin comprises an amino acid sequence as shown in SEQ ID NO: 1 or SEQ ID NO: 3.

In certain preferred embodiments, the metal in the metal chelate affinity chromatography column of the present disclosure has an ion form of Ni²⁺, Cu²⁺, Co²⁺, or Zn²⁺.

In some preferred specific embodiments, the variants have the following amino acid residue differences compared to SEQ ID NO: 1: C869S/A, and/or, C1093S/A;
preferably, the amino acid residue differences are selected from the group consisting of:
(1) C869S;
(2) C869A;
(3) C869S and C1093S;
(4) C869A and C1093A.

In addition, the amino acid residue differences can also be selected from the group consisting of:
(5) C1093S;
(6) C1093A;
(7) C869S and C1093A;
(8) C869A and C1093S.

More preferably, a nucleotide sequence encoding the tetanus toxin is shown in SEQ ID NO: 2.

The variants enumerated in the present disclosure are merely illustrative and are intended to demonstrate that as long as the core sequence of the tetanus toxin remains unchanged, the tetanus toxin can be purified by the method of the present disclosure, and the technical effects of high purity of target product, as well as simple and rapid purification steps can be obtained. Any technical solutions that only change individual amino acid residues of the tetanus toxin, such as those at C-terminus or N-terminus, without making substantial and creative changes to the method of purification of the present disclosure, still fall within the scope of protection of the present disclosure.

The medium of the metal chelate affinity chromatography column is preferably Ni-NTA, Ni Focurose 6FF, IDA-Focurose 6FF, IMAC Focurose, or Chelating Sepharose FF.

In certain preferred embodiments, the method of purification comprises steps of column equilibration, sample loading, washing, and elution, and a metal chelate affinity chromatography eluate is obtained after the elution.

Preferably, an equilibration buffer used for the column equilibration or the washing is PB with a pH of 6 to 8, e.g., 7.5, or Tris-HCl buffer with a pH of 6 to 10, e.g., 8.

More preferably, 3 to 7 column volumes, e.g., 5 column volumes of the equilibration buffer is used for the column equilibration or the washing.

When loading the sample, the metal chelate affinity chromatography column has a loading capacity of preferably 5 mg/mL to 20 mg/mL, e.g., 8 mg/mL, 10 mg/mL, 12 mg/mL, 14 mg/mL, 16 mg/mL, or 18 mg/mL.

In certain preferred embodiments, a buffer used in the elution is a biocompatible buffer containing 10 to 50 mM imidazole, e.g., 30 mM imidazole; the biocompatible buffer is preferably PB or Tris-HCl.

Preferably, the method of purification further comprises a step of purification using an anion exchange chromatography column.

More preferably, the purification includes steps of column equilibration, sample loading, washing, and elution.

In certain preferred embodiments, the steps as described above include:
(1) equilibrating the anion exchange chromatography column using an anion equilibration buffer, which is PB with a concentration of 5 to 50 mM and a pH of 6 to 8, e.g., PB with a concentration of 10 mM and a pH of 7.5;
(2) loading the obtained metal chelate affinity chromatography eluate onto the column;
(3) washing with the anion equilibration buffer from step (1) and rinsing with buffer A, which contains 10 mM PB and 40 mM NaCl and has a pH of 6 to 8, e.g., 7.5;
(4) eluting with buffer B and collecting the product. The buffer B contains 10 mM PB and 80 mM NaCl and has a pH of 6 to 8, e.g., 7.5.

In the above step (1), a usage amount of the anion equilibration buffer is preferably 3 to 7 column volumes, e.g., 5 column volumes;
in the above step (3), a usage amount of the buffer A is preferably 1 to 5 column volumes, e.g., 3 column volumes;
in the above step (4), a usage amount of the buffer B is preferably 1 to 5 column volumes, e.g., 3 column volumes.

In certain preferred embodiments, the anion exchange chromatography column is a GE DEAE FF anion exchange chromatography column or a GE Q HP anion chromatography column.

Another aspect of the present disclosure provides an application of metal chelate affinity chromatography in the purification of tetanus toxin or variants thereof, and the ion form of the metal is, for example, Ni²⁺, Cu²⁺, Co²⁺, or Zn²⁺; the tetanus toxin does not contain an affinity tag; the variants are as defined in the first aspect of the present disclosure.

Preferably, the tetanus toxin comprises an amino acid sequence as shown in SEQ ID NO: 1 or SEQ ID NO: 3.

The TTr protein of the present disclosure is a protein corresponding to fragment C at the C-terminus of the heavy chain of tetanus toxin, i.e., tetanus toxin fragment C, and its corresponding amino acid sequence is shown in SEQ ID NO: 1.

The tetanus toxin of the present disclosure has an amino acid sequence as shown in SEQ ID NO: 3.

The amino acid sites corresponding to C869S, C869A, C1093S, or C1093A of the present disclosure are numbered based on the complete tetanus toxin protein sequence (i.e., the sequence as shown in SEQ IN NO: 3), for example, the amino acid at position 869 in SEQ ID NO: 3 is cysteine (Cys, C).

On the basis of common knowledge in the art, the above preferred conditions can be arbitrarily combined to obtain preferred examples of the present disclosure.

The purification process for tetanus toxin, TTr protein, and variants thereof of the present disclosure comprises: firstly, rapidly capturing the target protein using metal chelate affinity chromatography, followed by further removing product-related impurities, such as impure proteins, nucleic acids, and bacterial endotoxins, using anion exchange chromatography. This method achieves rapid purification and preparation of high-purity target proteins, obtains high recovery rate, greatly reduced cost, and is conducive to large-scale production of the target proteins. It has the following advantages:
(1) simplify the purification process for tetanus toxin, TTr protein, or variants thereof, and reduce the production cost (the two mediums used in the present disclosure have low cost, long service life, high loading capacity, and high purification recovery rate).
(2) improve the purity of tetanus toxin, TTr protein, or variants thereof.
(3) improve the purification recovery rate of tetanus toxin, TTr protein, or variants thereof.

The reagents and raw materials used in the present disclosure are all commercially available.

The positive and progressive effects of the present disclosure are:
the present disclosure uses biocompatible agarose-based nickel affinity chromatography and anion exchange chromatography as separation subjects, which can quickly and effectively remove impure proteins and enrich target proteins; it improves the separation and purification efficiency of protein and greatly reduces the loss of proteins.

### BRIEF DESCRIPTION OF THE DRAWINGS

FIG. 1 shows the schematic diagram of the recombinant expression plasmid pET21-TTr.

FIG. 2 shows the electropherogram of metal ion screening of TTr protein purified by metal chelate affinity chromatography, wherein 1 represents supernatant of lysate; 2 represents Ni²⁺ flow-through; 3 represents Ni²⁺ elution; 4 represents Cu²⁺ flow-through; 5 represents Cu²⁺ elution; 6 represents Co²⁺ flow-through; 7 represents Co²⁺ elution; 8 represents Zn²⁺ flow-through; 9 represents Zn²⁺ elution; 10 represents protein marker.

FIG. 3 shows the nickel column chromatographic profile of TTr protein.

FIG. 4 shows the anion exchange chromatographic profile of TTr protein.

FIG. 5 shows the electropherogram of samples during the purification process of TTr protein, wherein 1 represents protein marker; 2 represents supernatant of lysate; 3 represents post-nickel column; 4 represents post-anion column; 5 represents final purified sample.

FIG. 6 shows the HPLC spectrum of TTr protein.

FIG. 7 shows the electropherogram of nickel column chromatography of tetanus toxin; 1 represents protein marker; 2 represents pre-nickel column (non-reduced); 3 represents pre-nickel column (reduced); 4 represents flow-through (non-reduced); 5 represents flow-through (reduced); 6 represents post-nickel column (non-reduced); 7 represents post-nickel column (reduced); 8 represents post-nickel column and ion exchange (non-reduced); 9 represents post-nickel column and ion exchange (reduced); 10 represents protein marker.

FIG. 8 shows the electropherogram of TTr protein variants after purification; 1 represents TTr protein variant (C869S); 2 represents TTr protein variant (C869A); 3 represents TTr protein variants (C869S and C1093S); 4 represents TTr protein variants (C869A and C1093A); 5 represents protein marker.

### DETAILED DESCRIPTION OF THE PREFERRED EMBODIMENT

The present disclosure is further illustrated below by means of examples, but it is not thereby limited to the scope of the examples. The experimental methods for which the specific conditions are not specified in the following examples shall be selected according to the conventional methods and conditions, or according to the commodity instructions.

### Example 1: Sequence Optimization and Construction of Expression Vector of TTr Protein

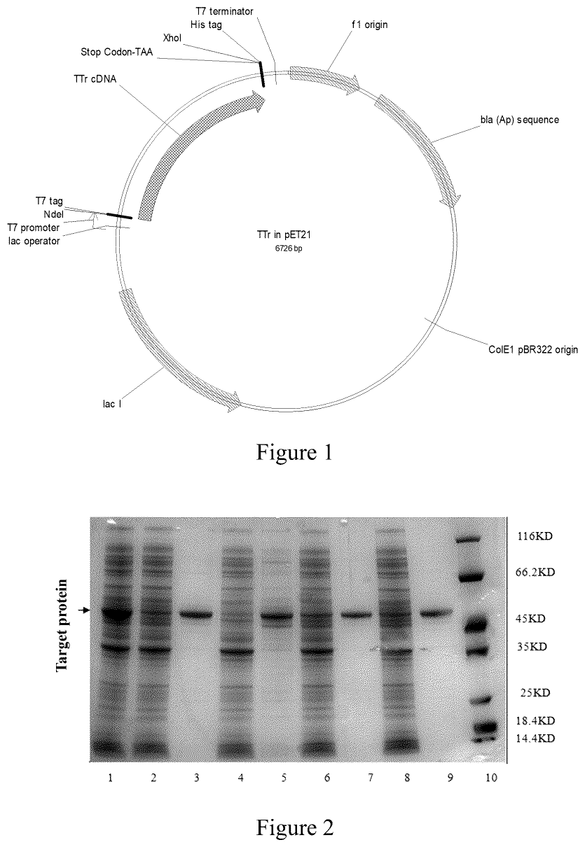

The reported gene and amino acid sequences of tetanus toxin fragment C (TTr protein) were queried by NCBI. A target gene with a stop codon (TAA) was synthesized after codon optimization of the target gene sequence of TTr protein (SEQ ID NO: 2). The target gene was then subjected to amplification and purification using PCR technology. After double enzyme digestion (with the restriction sites of NdeI and XhoI), the target gene fragment with the stop codon was cloned into the protein expression vector pET21a (+) between restriction endonucleases NdeI and XhoI (pET21-TTr, as shown in FIG. 1). Due to the presence of the stop codon, the His tag in pET21a (+) was not expressed. The recombinant plasmid was transformed into E. coli Top10 competent cells (cloning host bacteria), and positive transformants were selected, which were correctly identified by PCR and double enzyme digestion of the recombinant plasmid, and the gene sequencing results were consistent with the optimized target gene.

### Example 2: Expression and Identification of TTr Protein in E. coli

The plasmid pET21-TTr was transformed into E. coli BL21 (DE3) competent cells. A single colony was selected and incubated in 10 mL of LB (Amp) liquid medium at 37° C. with shaking at 250 rpm until OD₆₀₀ reached 0.8, then added with 0.1 mM IPTG, and incubated at 25° C. with shaking at 250 rpm for 4 hours. The culture medium was centrifuged at 8000 rpm and 4° C. to collect the bacterial cells, which were then resuspended in PBS and subjected to sonication for lysis. The lysate was centrifuged at 8000 rpm and 4° C. to collect the supernatant, and the pellet was resuspended in PBS. SDS-PAGE electrophoresis was performed for comparison using an empty vector expression product as a control. TTr protein was expressed in a soluble form with a molecular weight of approximately 50 kDa, which met the expected standards. The molecular weight of TTr protein was further identified by mass spectrometry, verifying the correct expression of TTr protein.

### Example 3: Fermentative Expression of TTr Protein

300 mL of seed culture of TTr protein expressed by shake flask was transferred to a 10 L fermenter, incubated until OD₆₀₀ reached 20, then added with 0.5 mM IPTG, and incubated at 25° C. with shaking at 300 rpm for 20 hours. The culture medium was centrifuged at 8000 rpm and 4° C. to collect the bacterial cells, which were then resuspended in PB buffer (pH 7.5) at a concentration of 10% (w/v) and subjected to homogeneous lysis. The lysate was centrifuged at 8000 rpm and 4° C. to collect the supernatant.

### Example 4: Purification of TTr Protein by Metal Chelate Affinity Chromatography

Preparation of metal ion affinity chromatography column: Seven 5 mL IDA-Focurose 6FF pre-packed columns (Huiyan Bio) were taken and washed with 5 column volumes of 0.1 M NiSO₄·6H₂O, 0.1 M CuSO₄·5H₂O, 0.1 M Co(NO₃)₂·6H₂O, 0.1 M ZnSO₄, 0.1 M MgSO₄, 0.1 M FeCl₃, and 0.1 M MnCl₂ solutions at a flow rate of 3 mL/min, respectively, the arm IDA (iminodiacetic acid) of IDA-Focurose 6FF was chelated with Ni²⁺, Cu²⁺, Co²⁺, Zn²⁺, Mg²⁺, Fe²⁺, and Mn²⁺, respectively, to prepare Ni²⁺-IDA-Focurose 6FF, Cu²⁺-IDA-Focurose 6FF, Co²⁺-IDA-Focurose 6FF, Zn²⁺-IDA-Focurose 6FF, Mg²⁺-IDA-Focurose 6FF, Fe³⁺-IDA-Focurose 6FF, and Mn²⁺-IDA-Focurose 6FF.

The prepared immobilized metal ion affinity chromatography columns were washed with 10 column volumes of ultrapure water to remove unbound metal ions, then equilibrated with 5 column volumes of PB buffer, pH 7.5, and 20 mL of the supernatant containing TTr protein was loaded onto the column at a flow rate of 3 mL/min. After loading the sample, the column was equilibrated with 5 column volumes of equilibration buffer, then eluted with PB buffer containing 30 mM imidazole to collect an eluate based on the main peak at UV 280 nm, and finally washed with 0.5 M imidazole and purified water, respectively.

Experimental results: the chromatography columns chelated with four metal ions, Ni²⁺, Cu²⁺, Co²⁺, and Zn²⁺, respectively, have specific affinity adsorption for TTr protein, and the adsorption of impure proteins can be effectively removed by washing, while TTr protein can be specifically adsorbed onto the chromatography columns. The purity of the supernatant containing TTr protein can reach more than 80% after purification by Cu²⁺-IDA-Focurose 6FF, while the purity of the supernatant of lysate containing TTr protein can reach more than 95% after purification by Ni²⁺/Co²⁺/Zn²⁺-IDA-Focurose 6FF; when purifying TTr protein using the metal ion chelate affinity chromatography column, the TTr protein is specifically adsorbed with a recovery rate of over 90%. The results are shown in Table 1. The affinity chromatography columns chelated with three metal ions, Mg²⁺, Fe³⁺, and Mn²⁺, respectively, have no affinity interaction with TTr protein, and were not effective in purification. As shown in FIG. 2.

TABLE 1
Summary of purification effect of TTr protein by
affinity chromatography with different metal ions

| Sample | Purity (%) | Recovery rate (%) |
|---|---|---|
| Supernatant of lysate containing | 29.87% | N/A |
| TTr protein | | |
| Ni²⁺-IDA Focurose 6FF | 95.20% | 95.60% |
| Cu²⁺-IDA Focurose 6FF | 81.03% | 96.08% |
| Co²⁺-IDA Focurose 6FF | 96.11% | 94.40% |
| Zn²⁺-IDA Focurose 6FF | 95.72% | 95.71% |
| Mg²⁺-IDA Focurose 6FF | 30.28% | 97.22% |
| Fe³⁺-IDA Focurose 6FF | 29.57% | 95.83% |
| Mn²⁺-IDA Focurose 6FF | 31.08% | 96.94% |

### Example 5: Purification of TTr Protein by Anion Exchange Chromatography

TTr protein can be further purified by anion exchange chromatography after purification by metal ion affinity chromatography. A GE DEAE FF anion exchange chromatography column with a column volume of 5 mL was selected. The chromatography column was equilibrated with 5 column volumes of anion equilibration buffer (10 mM PB, pH 7.5), and then a sample of the eluate collected from metal ion affinity chromatography was loaded onto the column. After loading the sample, the chromatography column was washed with 5 column volumes of equilibration buffer, then rinsed with 3 column volumes of a buffer (10 mM PB, 40 mM NaCl, pH 7.5), and finally eluted with a buffer (10 mM PB, 80 mM NaCl, pH 7.5) to collect TTr protein based on the main peak at UV 280 nm. The chromatography column was regenerated with 3 column volumes of 1 M NaCl, then washed with purified water until the conductivity was stable, and the column was stored in 20% ethanol. The results are shown in Table 2.

TABLE 2
Summary of purification effect of TTr protein
by anion exchange chromatography columns

| Purification conditions | Purity (%) | Recovery rate (%) |
|---|---|---|
| Ni²⁺-IDA Focurose 6FF + GE DEAE FF | 99.51% | 82.25% |
| Cu²⁺-IDA Focurose 6FF + GE DEAE FF | 98.07% | 78.08% |
| Co²⁺-IDA Focurose 6FF + GE DEAE FF | 99.35% | 80.19% |
| Zn²⁺-IDA Focurose 6FF + GE DEAE FF | 99.64% | 83.02% |

### Example 6: Purification and Characterization of TTr Protein

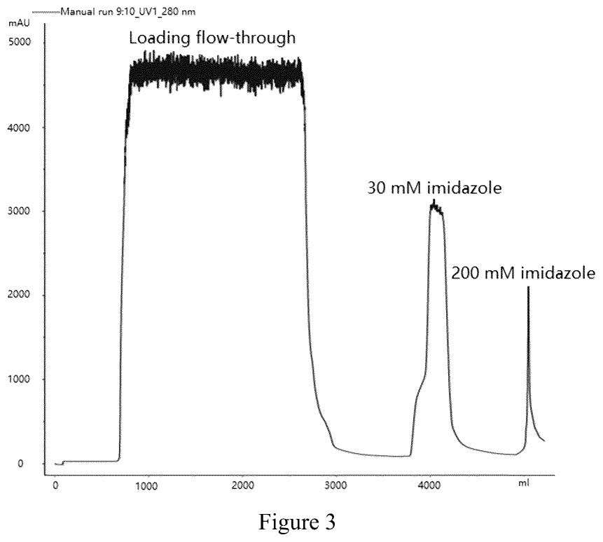

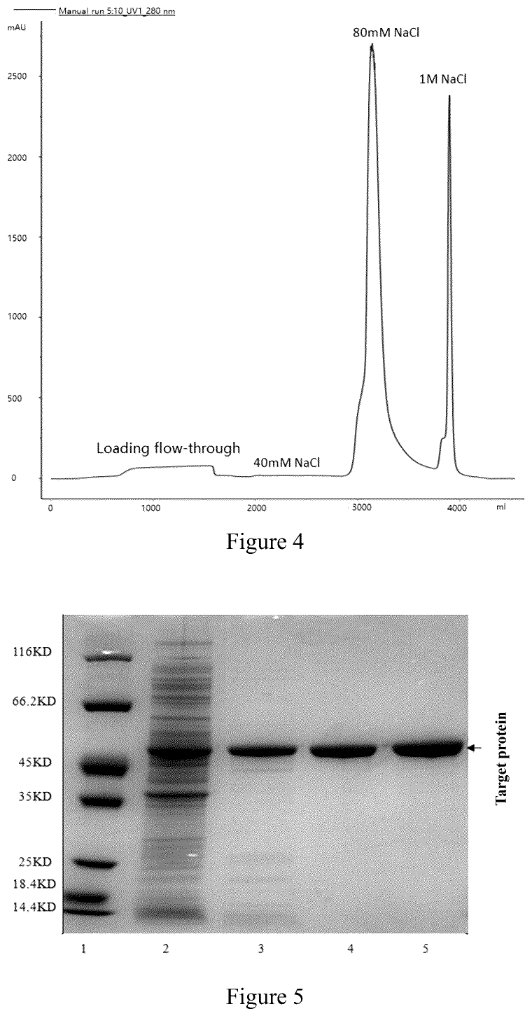

According to the methods described in Example 4 and Example 5, the recombinantly expressed TTr protein was purified and analyzed by Ni affinity chromatography in a scale-up purification procedure. TTr protein was first purified by 200 mL Ni-IDA ion affinity chromatography with a buffer containing 30 mM imidazole (as shown in FIG. 3). The sample was then further purified by anion exchange chromatography (GE DEAE FF 200 mL) and eluted with a buffer (10 mM PB, 80 mM NaCl, pH 7.5), as shown in FIG. 4.

The purification results of each step are shown in Table 3 below. After metal chelate affinity chromatography, the purity of the sample was approximately 95.20%. After further purification by anion exchange chromatography, the purity was elevated to 99.78%, and a final recovery rate of the entire purification process was 84.30%.

TABLE 3
Summary table of TTr protein purification

| | | Protein | | | |
|---|---|---|---|---|---|
| | | concen- | | Target | Recovery |
| | Volume | tration | Purity | protein | rate |
| Step | (mL) | (mg/mL) | (%) | (mg) | (%) |
| Supernatant | 2000 | 9.55 | 29.47 | 5629 | N/A |
| of lysate | | | | | |
| Ni-IDA | 950 | 5.77 | 95.20 | 5218 | 92.71 |
| chromatography | | | | | |
| GE DEAE FF | 1500 | 3.17 | 99.78 | 4745 | 90.93 |
| chromatography | | | | | |

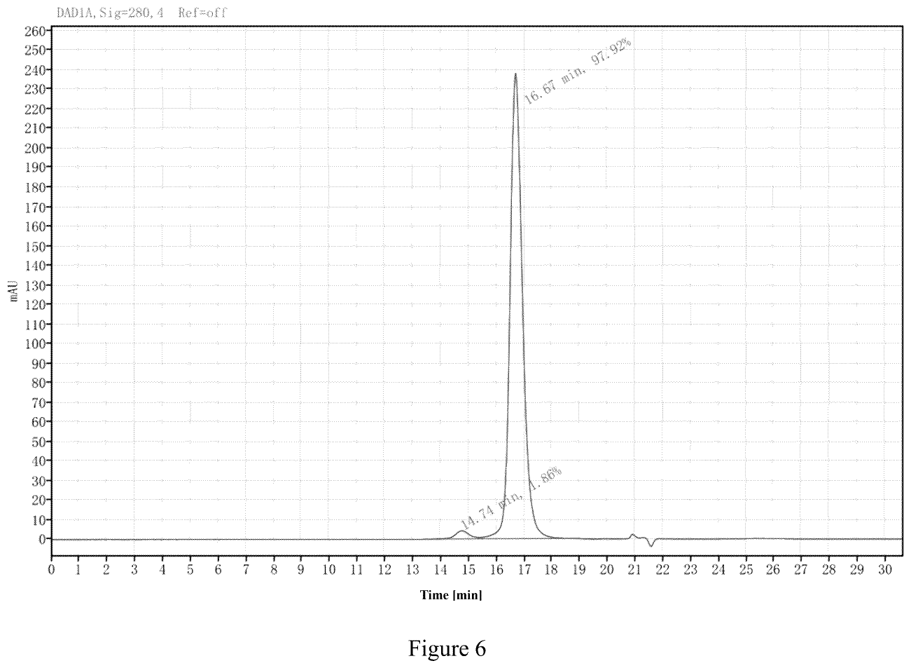

Electrophoresis and SEC-HPLC analysis were performed on the purified TTr protein. FIG. 5 shows the SDS-PAGE electropherogram of the samples from the purification process of TTr protein, indicating that a very high purity can be achieved by one-step purification using Ni chromatography. SEC-HPLC analysis was performed on the purified sample, and the results showed good peak shape symmetry in HPLC and good sample homogeneity, wherein TTr protein mainly existed in the form of monomers, accounting for 97.92%, and it also contains dimers, accounting for approximately 1.86% (as shown in FIG. 6).

### Example 7: Fermentation and Purification of Tetanus Toxin

The seed culture obtained by incubating the tetanus strain (ATCC 10779) was transferred to a 5 L fermenter, fermented statically at 35° C. for approximately 96 hours, and then incubated with stirring for 68 hours under continuous aeration (1 L/min). The fermentation broth was centrifuged at 9000 rpm for 30 minutes to collect the supernatant.

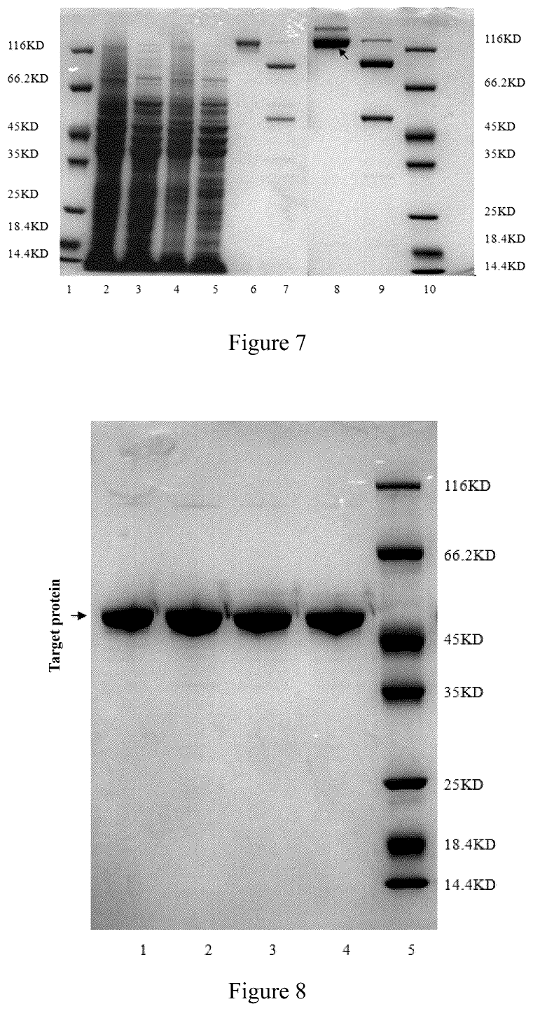

The supernatant was treated with 30 KDa membrane for buffer exchange and concentration, and then purified by affinity chromatography using IDA-Ni Focurose 6FF. The column was first equilibrated with 20 mM Tris-HCl buffer (pH 8.0) and then eluted with Tris-HCl buffer containing 25 mM imidazole. The eluate containing the target protein was passed through a GE Q HP chromatography column for anion exchange purification. Impure proteins were eluted with Tris-HCl buffer (pH 8.0) containing 200 mM NaCl. Tetanus toxin protein was eluted with Tris-HCl buffer (pH 8.0) containing 250 mM NaCl and collected. The remaining steps were similar to those described in Example 5. After two-step purification by chromatography, tetanus toxin protein with a purity of up to 99% was obtained from untagged tetanus toxin (as shown in FIG. 7).

### Example 8: Prokaryotic Soluble Expression of TTr Protein Variant (C869S)

Using the synthesized gene fragment encoding TTr protein as a template, the upstream primer F1 (SEQ ID NO: 4): 5′-TTAAGAAGGAGATATACATATGAAAAACCTTGACAGCTGGGTGGATAACGAAGAG G-3′ (NdeI restriction site was shown in underline, and mutated codon was shown in boldface) and the downstream primer R1 (SEQ ID NO: 5): 5′-GGTGGTGGTGGTGGTGCTCGAGTTAGTCGTTCGTCCACC-3′ (XhoI restriction site was shown in underline) were designed. DNA polymerase was used for PCR amplification, thereby introducing a mutation (Cys869 (TGC) to Ser869 (AGC)) in the TTr protein fragment to obtain a complete DNA fragment of TTr protein variant (C869S). The DNA fragment of TTr protein variant (C869S) was recovered and ligated into the pET21a (+) vector double-digested by NdeI and XhoI via homologous recombination, then transformed into E. coli Top10 cells by heat shock, and positive clones were selected for sequencing. The correctly sequenced expression vector of TTr protein variant (C869S) was transformed into E. coli BL21 (DE3) cells. A single colony was selected and incubated in 50 mL of LB liquid medium at 37° C. with shaking at 250 rpm until OD₆₀₀ reached 0.8, then added with IPTG (final concentration of 0.1 mM), and the expression was induced at 25° C. with shaking at 250 rpm for 16 hours. The bacterial pellet was collected by centrifugation, resuspended in PBS and subjected to sonication for lysis, then the supernatant was collected by centrifugation, and SDS-PAGE protein electrophoresis was performed to detect the expression of the target protein.

### Example 9: Prokaryotic Soluble Expression of TTr Protein Variant (C869a)

Using the synthesized gene fragment encoding TTr protein as a template, the upstream primer F2 (SEQ ID NO: 6): 5′-TTAAGAAGGAGATATACATATGAAAAACCTTGACGCCTGGGTGGATAACGAAGAG G-3′ (NdeI restriction site was shown in underline, and mutated codon was shown in boldface) and the downstream primer R1 (SEQ ID NO: 5): 5′-GGTGGTGGTGGTGGTGCTCGAGTTAGTCGTTCGTCCACC-3′ (XhoI restriction site was shown in underline) were designed. DNA polymerase was used for PCR amplification, thereby introducing a mutation (Cys869 (TGC) to Ala869 (GCC)) in the TTr protein fragment to obtain a complete DNA fragment of TTr protein variant (C869A). The DNA fragment of TTr protein variant (C869A) was recovered and ligated into the pET21a (+) vector double-digested by NdeI and XhoI via homologous recombination, then transformed into E. coli Top10 cells by heat shock, and positive clones were selected for sequencing. The correctly sequenced expression vector of TTr protein variant (C869A) was transformed into E. coli BL21 (DE3) cells. A single colony was selected and incubated in 50 mL of LB liquid medium at 37° C. with shaking at 250 rpm until OD₆₀₀ reached approximately 0.8, then added with IPTG (final concentration of 0.1 mM), and the expression was induced at 25° C. with shaking at 250 rpm for 16 hours. The bacterial pellet was collected by centrifugation, resuspended in PBS and subjected to sonication for lysis, then the supernatant was collected by centrifugation, and SDS-PAGE protein electrophoresis was performed to detect the expression of the target protein.

### Example 10: Prokaryotic Soluble Expression of TTr Protein Variants (C869S and C1093S)

Using the synthesized gene fragment encoding TTr protein as a template, the following upstream primer and downstream primer were designed: F1 (SEQ ID NO: 4): 5′-TTAAGAAGGAGATATACATATGAAAAACCTTGACAGCTGGGTGGATAACGAAGAG G-3′ (NdeI restriction site was shown in underline, and mutated codon was shown in boldface); R2 (SEQ ID NO: 10): 5′-CCTTCGGATTTAACGCCTTACTAAATATCCGAAAC-3′. PCR amplification was used to obtain partial fragments of TTr protein variants (C869S and C1093S), thereby introducing a mutation (Cys869 (TGC) to Ser869 (AGC)) in the TTr protein fragment. The following upstream primer and downstream primer were designed: F3 (SEQ ID NO: 7): 5′-GTTTCGGATATTTAGTAAGGCGTTAAATCCGAAGG-3′ (mutated codon was shown in boldface); R1 (SEQ ID NO: 5): 5′-GGTGGTGGTGGTGGTGCTCGAGTTAGTCGTTCGTCCACC-3′ (XhoI restriction site was shown in underline). PCR amplification was used to obtain partial fragments of TTr protein variants (C869S and C1093S), thereby introducing a mutation (Cys1093 (TGT) to Ser1093 (AGT)) in the TTr protein fragments. The two DNA fragments were subjected to gel recovery and purification, and ligated into the pET21a (+) vector double-digested by NdeI and XhoI via homologous recombination, then transformed into E. coli Top10 cells by heat shock, and positive clones were selected for sequencing. The correctly sequenced expression vectors of TTr protein variants (C869S, C1093S) were transformed into E. coli BL21 (DE3) cells, respectively. A single colony was selected and incubated in 50 mL of LB liquid medium at 37° C. with shaking at 250 rpm until OD₆₀₀ reached approximately 0.8, then added with IPTG (final concentration of 0.1 mM), and the expression was induced at 25° C. with shaking at 250 rpm for 16 hours. The bacterial pellet was collected by centrifugation, resuspended in PBS and subjected to sonication for lysis, then the supernatant was collected by centrifugation, and SDS-PAGE protein electrophoresis was performed to detect the expression of the target protein.

### Example 11: Prokaryotic Soluble Expression of TTr Protein Variants (C869A and C1093A)

Using the synthesized gene fragment encoding TTr protein as a template, the following upstream primer and downstream primer were designed: F2 (SEQ ID NO: 6): 5′-TTAAGAAGGAGATATACATATGAAAAACCTTGACGCCTGGGTGGATAACGAAGAG G-3′ (NdeI restriction site was shown in underline, and mutated codon was shown in boldface); R3 (SEQ ID NO: 8): 5′-CCTTCGGATTTAACGCCTTGGCAAATATCCGAAAC-3′. PCR amplification was used to obtain partial fragments of TTr protein variants (C869A and C1093A), thereby introducing a mutation (Cys869 (TGC) to Ala869 (GCC)) in the TTr protein fragments. The following upstream and downstream primers were designed: F4 (SEQ ID NO: 9): 5′-GTTTCGGATATTTGCCAAGGCGTTAAATCCGAAGG-3′ (mutated codons were shown in boldface); R1 (SEQ ID NO: 5): 5′-GTGGTGGTGGTGGTGCTCGAGTTAGTCGTTCGTCCACC-3′ (Xhol restriction site was shown in underline). PCR amplification was used to obtain partial fragments of TTr protein variants (C869A and C1093A), thereby introducing a mutation (Cys1093 (TGT) to Ala1093 (GCC)) in the TTr protein fragments. The two DNA fragments were subjected to gel recovery and purification, and ligated into the pET24a (+) vector double-digested by NdeI and XhoI via homologous recombination, then transformed into E. coli Top10 cells by heat shock, and positive clones were selected for sequencing. The correctly sequenced expression vectors of TTr protein variants (C869A and C1093A) were transformed into E. coli BL21 (DE3) cells, respectively. A single colony was selected and incubated in 50 mL of LB liquid medium at 37° C. with shaking at 250 rpm until OD₆₀₀ reached approximately 0.8, then added with IPTG (final concentration of 0.1 mM), and the expression was induced at 25° C. with shaking at 250 rpm for 16 hours. The bacterial pellet was collected by centrifugation, resuspended in PBS and subjected to sonication for lysis, then the supernatant was collected by centrifugation, and SDS-PAGE protein electrophoresis was performed to detect the expression of the target protein.

### Example 12: Fermentation and Purification of TTr Protein Variants

200 mL each of seed culture of expressed TTr protein variant (C869S), TTr protein variant (C869A), TTr protein variants (C869S and C1093S), and TTr protein variants (C869A and C1093A) was transferred to a 5 L fermenter, respectively, incubated until OD₆₀₀ reached approximately 2, then added with IPTG (final concentration of 0.5 mmol/L), and incubated at 25° C. with shaking at 300 rpm for 24 hours. The bacterial cells were collected by centrifugation at 10000×g and 4° C., resuspended in 20 mmol/L Tris-HCl buffer (pH 8.0), and subjected to homogeneous lysis. The supernatant was collected by centrifugation at 20000×g and 4° C. The supernatant was purified by affinity chromatography using IDA-Ni Focurose 6FF. The column was first equilibrated with 20 mM Tris-HCl buffer (pH 8.0) and then eluted with Tris-HCl buffer containing 25 mM imidazole. The eluate containing the target protein was passed through a GE Q HP chromatography column for anion exchange purification. Impure proteins were eluted with Tris-HCl buffer (pH 8.0) containing 40 mM NaCl. Target proteins were eluted with Tris-HCl buffer (pH 8.0) containing 100 mM NaCl and collected. The remaining purification steps were similar to those described in Example 5. After two-step purification by chromatography, untagged TTr protein variant (C869S), TTr protein variant (C869A), TTr protein variants (C869S and C1093S), and TTr protein variants (C869A and C1093A) were obtained with up to 99% purity (as shown in FIG. 8).

What is claimed is:

1. A method for purifying tetanus toxin or variants thereof, comprising using metal chelate affinity chromatography to purify tetanus toxin or variants thereof, wherein the tetanus toxin does not contain an affinity tag.

2. The method according to claim 1, wherein the metal has an ion form of Ni²⁺, Cu²⁺, Co²⁺, or Zn²⁺.

3. The method according to claim 2, wherein the tetanus toxin comprises an amino acid sequence as shown in SEQ ID NO: 1 or SEQ ID NO: 3.

4. The method according to claim 1, wherein the variants have the following amino acid residue differences compared to SEQ ID NO: 1: C869S/A or C1093S/A.

5. The method according to claim 4, wherein the amino acid residue differences are selected from the group consisting of:
(1) C869S;
(2) C869A;
(3) C869S and C1093S;
(4) C869A and C1093A.

6. The method according to claim 5, wherein the nucleotide sequence encoding the tetanus toxin is shown in SEQ ID NO: 2.

7. The method according to claim 1, wherein the medium of the metal chelate affinity chromatography column is Ni-NTA, Ni Focurose 6FF, IDA-Focurose 6FF, IMAC Focurose, or Chelating Sepharose FF.

8. The method according to claim 1, comprising steps of column equilibration, sample loading, washing, and elution, and a metal chelate affinity chromatography eluate is obtained after the elution.

9. The method according to claim 8, wherein an equilibration buffer used for the column equilibration or the washing is PB with a pH of 6 to 8, for example, 7.5, or Tris-HCl buffer with a pH of 6 to 10, for example, 8.

10. The method according to claim 9, wherein 3 to 7 column volumes, for example, 5 column volumes of the equilibration buffer is used for the column equilibration or the washing.

11. The method according to claim 8, wherein the metal chelate affinity chromatography column has a loading capacity of 5 mg/mL to 20 mg/mL, when loading the sample.

12. The method according to claim 8, wherein a buffer used in the elution is a biocompatible buffer containing 10 to 50 mM imidazole.

13. The method according to claim 12, wherein the biocompatible buffer contains 30 mM imidazole.

14. The method according to claim 13, wherein the biocompatible buffer is PB or Tris-HCl.

15. The method according to claim 1 further comprises a step of purification using an anion exchange chromatography column.

16. The method according to claim 15, wherein the purification using the anion exchange chromatography column comprises steps of column equilibration, sample loading, washing, and elution.

17. The method according to claim 16, wherein the purification using the anion exchange chromatography column comprises the steps of:
(1) equilibrating the anion exchange chromatography column using an anion equilibration buffer, which is PB with a concentration of 5 to 50 mM and a pH of 6 to 8, for example, PB with a concentration of 10 mM and a pH of 7.5;
(2) loading the obtained metal chelate affinity chromatography eluate onto the column;
(3) washing with the anion equilibration buffer from step (1) and rinsing with buffer A, which contains 10 mM PB and 40 mM NaCl and has a pH of 6 to 8, for example, 7.5;
(4) eluting with buffer B and collecting product, wherein the buffer B contains 10 mM PB and 80 mM NaCl and has a pH of 6 to 8, for example, 7.5.

18. The method according to claim 15, wherein in step (1), a usage amount of the anion equilibration buffer is 3 to 7 column volumes;
or in step (3), a usage amount of the buffer Ais 1 to 5 column volumes;
or in step (4), a usage amount of the buffer B is 1 to 5 column volumes.

19. The method according to claim 18, wherein in step (1), a usage amount of the anion equilibration buffer is 5 column volumes;
or in step (3), a usage amount of the buffer A is 3 column volumes;
or in step (4), a usage amount of the buffer B is 3 column volumes.

20. The method according to claim 19, wherein the anion exchange chromatography column is a GE DEAE FF anion exchange chromatography column or a GE Q HP anion exchange chromatography column.


SEQUENCE LISTING (10 sequences)

SEQ ID NO: 1 - protein, 451 aa, synthetic construct
  REGION 1..451: note = TTr protein
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 2 - DNA, 1353 bp, synthetic construct
  misc_feature 1..1353: note = Nucleotides of TTr protein
  source 1..1353: mol_type = other DNA
aaaaaccttgactgctgggtggataacgaagaggacatcgacgtcatattgaaaaaatcgactattttaaatttagacattaacaatgacattatttcggacatttcaggctttaactcatcagtaataacttatcctgacgcacaattggtccctggaatcaacggtaaagccatccatctggtgaacaacgaaagttccgaagtgattgtccataaagctatggacattgagtataacgatatgtttaataatttcactgtgagtttttggctgcgtgtccctaaagtatctgccagtcacttagagcaatatggtacgaacgaatactcgataatttcatctatgaaaaaacatagcttgtcaatcggctcaggctggtccgtctcactgaagggcaataatcttatctggaccctgaaggactctgcgggtgaagtacggcagatcacgtttagagatcttccggataaatttaacgcttatttagcgaacaaatgggtctttatcactatcacaaatgatcgtctgtcgagcgccaacctttatattaacggagtactgatgggtagtgctgagattaccggacttggagcgattcgggaagacaacaacatcacccttaaacttgatcgttgtaacaataataatcagtacgtgagcatcgacaagtttcggatattttgtaaggcgttaaatccgaaggaaattgaaaaattatatacatcgtatctgtcaatcactttcttgcgggatttctgggggaatcctcttcggtatgataccgaatactatcttatacctgtggcatctagtagtaaggatgtacagttgaagaacattacagattatatgtatcttaccaatgcacccagttacacaaacggcaagttaaatatctattatcgccggctttataacggtctgaaattcatcataaaaagatacactcccaacaacgagatagattcttttgtcaagagcggagacttcattaagttatatgtcagctacaacaacaatgagcacatagttggctacccaaaggacggaaacgcgtttaacaacctggatcgtatactgcgggtgggctataacgcgcctgggattcccttgtataaaaagatggaagcagtaaaattgcgcgacttgaagacgtactccgtacaacttaaactttacgacgacaaaaatgcgagtcttggattggttggtacgcacaacggacagatcgggaatgatccgaaccgtgacatcttaattgcatcaaattggtactttaatcatttgaaagacaaaatccttggctgcgattggtattttgttccgactgatgaggggtggacgaacgac

SEQ ID NO: 3 - protein, 1315 aa, synthetic construct
  REGION 1..1315: note = Tetanus toxin
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 4 - DNA, 56 bp, synthetic construct
  misc_feature 1..56: note = Upstream primer F1
  source 1..56: mol_type = other DNA
ttaagaaggagatatacatatgaaaaaccttgacagctgggtggataacgaagagg

SEQ ID NO: 5 - DNA, 39 bp, synthetic construct
  misc_feature 1..39: note = Downstream primer R1
  source 1..39: mol_type = other DNA
ggtggtggtggtggtgctcgagttagtcgttcgtccacc

SEQ ID NO: 6 - DNA, 56 bp, synthetic construct
  misc_feature 1..56: note = Upstream primer F2
  source 1..56: mol_type = other DNA
ttaagaaggagatatacatatgaaaaaccttgacgcctgggtggataacgaagagg

SEQ ID NO: 7 - DNA, 35 bp, synthetic construct
  misc_feature 1..35: note = Upstream primer F3
  source 1..35: mol_type = other DNA
gtttcggatatttagtaaggcgttaaatccgaagg

SEQ ID NO: 8 - DNA, 35 bp, synthetic construct
  misc_feature 1..35: note = Downstream primer R3
  source 1..35: mol_type = other DNA
ccttcggatttaacgccttggcaaatatccgaaac

SEQ ID NO: 9 - DNA, 35 bp, synthetic construct
  misc_feature 1..35: note = upstream primer F4
  source 1..35: mol_type = other DNA
gtttcggatatttgccaaggcgttaaatccgaagg

SEQ ID NO: 10 - DNA, 35 bp, synthetic construct
  misc_feature 1..35: note = Downstream primer R2
  source 1..35: mol_type = other DNA
ccttcggatttaacgccttactaaatatccgaaac